(19) United States
(12) Patent Application Publication
(10) Pub. No.: US 2015/0018721 A1
(43) Pub. Date: Jan. 15, 2015

(54) PRESSURE MONITORING SHOE

(71) Applicants: ADVANCED TECHNOLOGY DEVELOPMENT (SHENZHEN CO., LTD, Shenzhen (CN); ADVANPRO LIMITED, HK (CN)

(72) Inventors: Yangyong Wang, HK (CN); Guangfeng Wang, HK (CN)

(21) Appl. No.: 14/376,558

(22) PCT Filed: Oct. 31, 2012
(86) PCT No.: PCT/CN2012/083871

§ 371 Date: Aug. 4, 2014

(30) Foreign Application Priority Data
Feb. 16, 2012 (CN) 201210034726.8

(51) Int. Cl. A61B 5/00; A61B 5/103

(52) U.S. Cl. CPC A61B 5/6807; A61B 5/1038; A61B 5/7278; A61B 5/0004
USPC 600/592

(57) ABSTRACT

A pressure monitoring shoe comprises a main shoe body, a data processing device and at least two pressure sensors. The pressure sensors are arranged at one or a plurality of locations where the main shoe body is in contact with a foot, and the pressure sensor comprises a flexible textile sensor and an encapsulation device for encapsulating the flexible textile sensor. The data processing device is arranged in one or more of the sole space, the outside space of the shoe body or the rear space of the heel of the pressure monitoring shoe, and a signal connection line is arranged between the data processing device and the pressure sensor.

### CROSS-REFERENCE TO RELATED APPLICATIONS

This present application claims the benefit of priority to Chinese Patent Application NO. 201210034726.8 filed in Chinese Patent Office on Feb. 16, 2012 and entitled “PRESSURE MONITORING SHOE”, the content of which is hereby incorporated by reference in its entirety for all intended purposes.

### FIELD

The disclosure relates to the field of strain sensing, and particularly to a pressure monitoring shoe.

### BACKGROUND

No matter for the special professional groups, such as firemen or policemen; athletes of different games; or the aged people and patients, such as patients recuperated from stroke, and so on; it makes significant efforts that the pressure of different potions from a foot and the pressure distribution can be measured and detected rapidly, and the work efficiency, sport skills or recuperation for patients can be improved.

### SUMMARY

Exemplary embodiments of the present invention provide a pressure monitoring shoe, in which pressure from different potions of a foot can be rapidly monitored and detected.

One embodiment of the present invention provides a pressure monitoring shoe, comprising a main shoe body, a data processing device, and at least two pressure sensors; wherein, the pressure sensors are arranged at one or a plurality of locations where the main shoe body is in contact with a foot; and the data processing device is arranged in one or more of a sole space, an outside space of the shoe body or a rear area of the heel of the pressure monitoring shoe, and a signal connection line is arranged between the data processing device and the pressure sensor.

Wherein, the pressure sensor comprises a flexible textile sensor and an encapsulation device for encapsulating the flexible textile sensor, or the pressure sensor is denoted as a pressure sensor of resistance type, capacitive type, inductance type. The pressure data can be obtained by detecting a respective voltage or current output variation of the pressure sensor.

Wherein, the flexible textile sensor comprises a flexible textile layer having a sensing stress, a fixed layer bond on the flexible textile; no fixed layer is bond on a stress-sensing domain of the flexible textile, and the fixed layer is bond on a fixed domain besides the stress-sensing domain; a strain of the fixed layer is less than the strain of the flexible textile.

Wherein, the fixed layer is an inelastic mechanical textile.

Wherein, the flexible textile layer forms a C shape, and an upper arm and a lower arm of the C shape are the stress-sensing domains; the fixed layer is fixed on a starting point side, an ending point, and a left-bending side of the C shape; the strain of the fixed layer is less than the strain of the flexible textile.

Wherein, the data processing device comprises a transmitting module, which is configured to receive a signal generated by the pressure sensor and transmit the signal to an external data receiving platform by a wired or a wireless mode; and/or the data processing device comprises a data storage module, which is configured to receive and storage a signal generated by the pressure sensor, convert the signal generated by the pressure sensor into a pressure data, and storage the pressure data.

Wherein, the encapsulation device comprises a flexible shell body, the pressure sensor is encapsulated in the flexible shell body and fixed on a bottom substrate of the shell body, the flexible shell body is stretched with the flexible textile sensor being stretched and compressed with the flexible textile sensor being compressed; or/and the package device comprises a flexible coating, the flexible coating is coated on all sides of the pressure monitoring , and the flexible coating is stretched with the flexible textile sensor being stretched and compressed with the flexible textile sensor being compressed.

Wherein, a connecting electrical wire or a conductive button is bond, mechanically clamped, sewn on both sides of the stress-sensing domain in the strain direction; or the connecting electrical wire or the conductive button is bond, mechanically clamped, sewn on the fixed domain at the double sides of the stress-sensing domain in the strain direction, and the connecting electrical wire or the conductive button is conductively connected with the flexible textile.

In exemplary embodiments of the present invention, the pressure monitoring shoe can further comprise a charging module in the main shoe body, and the charging module is configured to charge for the pressure sensor and the data processing device. The charging module can be any one of a disposable un-chargeable battery, a chargeable battery, a mechanically charged device.

In exemplary embodiment of the present invention, the data processing device can be arranged in a sole space, an outside space of the shoe body or a rear area of the heel of the pressure monitoring shoe, which can make little inconvenience for a wearer, the sensor information can be obtained in time, and thus a data can be processed efficiently.

Furthermore, in exemplary embodiments of the present invention, the flexible textile sensor taken as the pressure sensor can be provided, the flexible textile sensor can be formed by binding little or no elastic fixed layer on the elastic stress-sensing flexible textile, and no fixed layer may be covered on the stress-sensing domain of the flexible textile. And thus, the stress-sensing domain of the flexible textile can sense the stress with only little or no deformation of other domains of the flexible textile. Therefore, it is convenient for the flexible textile to connect with other parts of the whole sensing system, and the accuracy of the sensor can be improved.

### BRIEF DESCRIPTION OF THE DRAWINGS

In order to illustrate the embodiments or existing technical solutions more clearly, a brief description of drawings that assists the description of embodiments of the invention or existing art will be provided below. It would be apparent that the drawings in the following description are only for some of the embodiments of the invention. A person having ordinary skills in the art will be able to obtain other drawings on the basis of these drawings without paying any creative work.

FIG. 1 is a top view of a pressure monitoring shoe showing where the locations of pressure sensors are arranged according to one embodiment of the invention;

FIG. 2 is a side view of a pressure monitoring shoe showing where the locations of pressure sensors are arranged in according to one embodiment of the invention;

FIG. 3 is a schematic view of a first location of a data processing device arranged in a pressure monitoring shoe according to one embodiment of the invention;

FIG. 4 is a schematic view of a second location of a data processing device arranged in a pressure monitoring shoe according to one embodiment of the invention;

FIG. 5 is a schematic view of a third location of a data processing device arranged in a pressure monitoring shoe according to one embodiment of the invention;

FIG. 6 is a front view of a composition of a flexible textile sensor according to one embodiment of the invention;

FIG. 7 is a side view of the flexible textile sensor described in FIG. 6;

FIG. 8 is a front view of another composition of a flexible textile sensor according to one embodiment of the invention;

FIG. 9 is a side view of the flexible textile sensor described in FIG. 8;

FIG. 10 is a specific structure diagram showing the connection of electrical wires on a flexible textile sensor according to one embodiment of the invention;

FIG. 11 is a specific structure diagram showing the connection of conductive buttons on a flexible textile sensor according to one embodiment of the invention;

FIG. 12 is a specific structure diagram of C shape flexible textile of a flexible textile sensor according to one embodiment of the invention;

FIG. 13 is a specific structure diagram of C shape flexible textile of a flexible textile sensor according to another embodiment of the invention;

FIG. 14 is a specific schematic diagram showing a hollow pattern in a semi-finished product of a flexible textile sensor.

### DETAILED DESCRIPTION OF ILLUSTRATED EMBODIMENTS

Further descriptions of present disclosure will be illustrated clearly and completely, which can be combined with embodiments in drawings and detailed embodiments. Obviously, the embodiments here are not all embodiments but only part of embodiments of present disclosure. Based on the embodiments of present disclosure, under premise of without paying out creative work, other embodiments obtained by the person having ordinary skill in the art are considered to encompass within the scope of the present invention.

One embodiment of the invention discloses a pressure monitoring shoe, which includes a main shoe body, a data processing device and at least two pressure sensors. The pressure sensors can be arranged in one or more locations where the main shoe body and a foot are connected (such as a location underneath the shoe insole, or anywhere in the shoe body); the data processing device can be arranged in one or more of a sole space, an outside space of the shoe body or a rear area of the heel of the pressure monitoring shoe, and a signal connection line is arranged between the data processing device and the pressure sensor.

Wherein, the pressure sensor comprises a flexible textile sensor and an encapsulation device which for encapsulating the flexible textile sensor, or the pressure sensor is denoted as a pressure sensor of resistance type, capacitive type, inductance type.

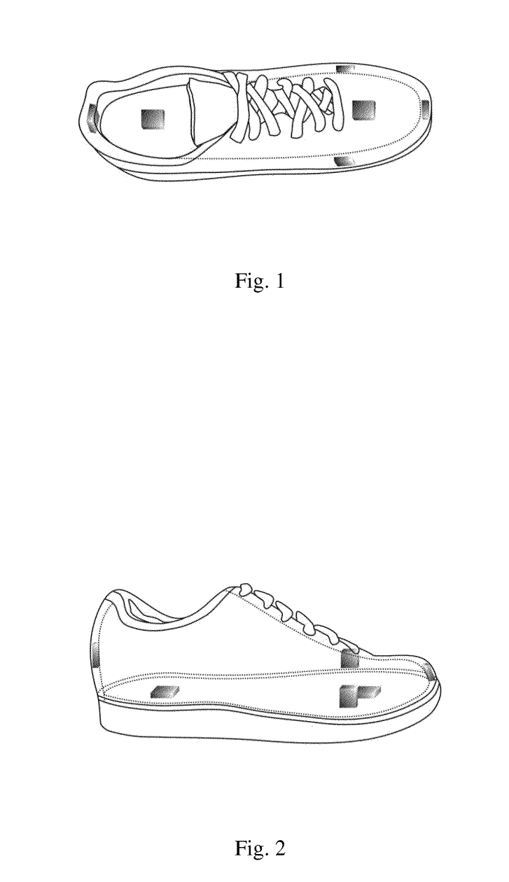

Referring to FIG. 1 and FIG. 2, it indicates the locations in the main shoe body where the pressure sensors are arranged. In one embodiment, the pressure sensors can be arranged in different locations, the grey cubicle shown in figures can be taken as a pressure sensor. The size of the cubicle shown in figures can only be used for illustration, and should not be limited to the virtual size of the pressure sensor.

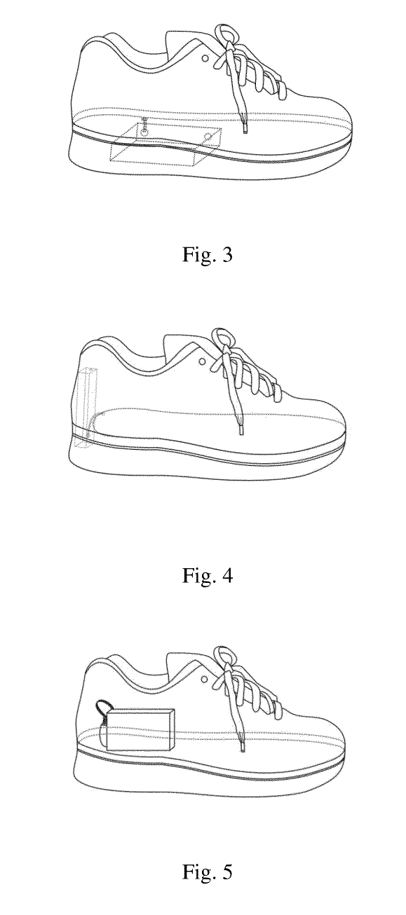

Referring to FIG. 3-FIG. 5, they indicate the different locations in the main shoe body where the data processing device is arranged. FIG. 3 indicates the data processing device is arranged in the shoe sole, FIG. 4 indicates the data processing device is arranged in the rear space of the shoe heel, and FIG. 5 indicates the data processing device is arranged in the outside space of the shoe body. And thus, the arrangement can make efficient use of the space existed in the shoe body, and can make the wearer feel no discomfort. Meanwhile, with the combination of the locations where the pressure sensors are arranged shown in FIG. 1 and FIG. 2, it can be more beneficial for a signal wire of the pressure sensor to connect with the data processing device.

Wherein, the data processing device comprises a transmitting module, which is configured to receive a signal generated by the pressure sensor and transmit the signal to an external data receiving platform by a wired or a wireless mode. Or the data processing device comprises a data storage module, which is configured to receive and storage a signal generated by the pressure sensor, convert the signal generated by the pressure sensor into a pressure data, and storage the pressure data.

Otherwise, the pressure monitoring shoe can further includes a charging module (not shown) arrange in the main shoe body (e.g. a location underneath the shoe insole, or any location in the shoe body), the charging module is configured to charge for the pressure sensor and the data processing device. In one particular implementation, the charging module can be any one of a disposable un-chargeable battery (such as a disposable button cell, or a dry cell), a chargeable battery (such as a lithium battery), or a mechanically charged device (such as a generator which converts the mechanical energy into power).

In order to detect a pressure signal better, in one embodiment of the invention, a flexible textile sensor for detecting the pressure signal can be applied for a specific implementation. The sensor can be encapsulated in an encapsulation device for waterproof, moisture proof, and for improving the service life and the accuracy of the sensor.

For example, the pressure sensor can be obtained by coating a flexible coating on the flexible textile sensor, which means the pressure sensor can include a flexible textile sensor and a flexible coating coated on all sides of the pressure sensor; and the flexible coating is stretched with the flexible textile sensor being stretched and compressed with the flexible textile sensor being compressed.

Meanwhile, the pressure sensor can be obtained by the manner of shell body encapsulation. In this way, the pressure sensor can comprise a flexible textile sensor and a flexible shell body which encapsulates the flexible textile sensor, the flexible textile sensor can be fixed on the bottom substrate of the shell body. The flexible shell body can be stretched with the flexible textile sensor being stretched and compressed with the flexible textile sensor being compressed. It means that the flexible shell body can perform with the compress/stretch process of the pressure sensor, and the sensitivity of the flexible textile sensor will not be influenced.

Particularly, the flexible shell body or the flexible coating can be a thermoplastic or a thermosetting polymer material; the bottom substrate material can be a normal polymer material by molding, injection molding or blow molding, and the like properties. For example, the flexible shell body or the flexible coating can be a thermoplastic elastomer material (such as EBS, SEBS and so on), a silicon rubber, a nature rubber, and all kinds of synthetic rubber. As the same, these materials can also be applied to the flexible coating.

It should be noticed that the two encapsulation manners above can be performed respectively to obtain the two kinds of pressure sensors above, and they can also be performed at the same time, which means the flexible textile sensor can be encapsulated into the flexible shell body after it is coated.

In one embodiment of the invention, the flexible textile sensor can be encapsulated to form the pressure sensor by the manner of coating or shelling. Due to the coated flexible coating or/and the flexible shell body may be made up of flexible materials, the sensitivity of the flexible textile sensor packaged in the materials may not be influenced, and the performance of the sensor can be improved.

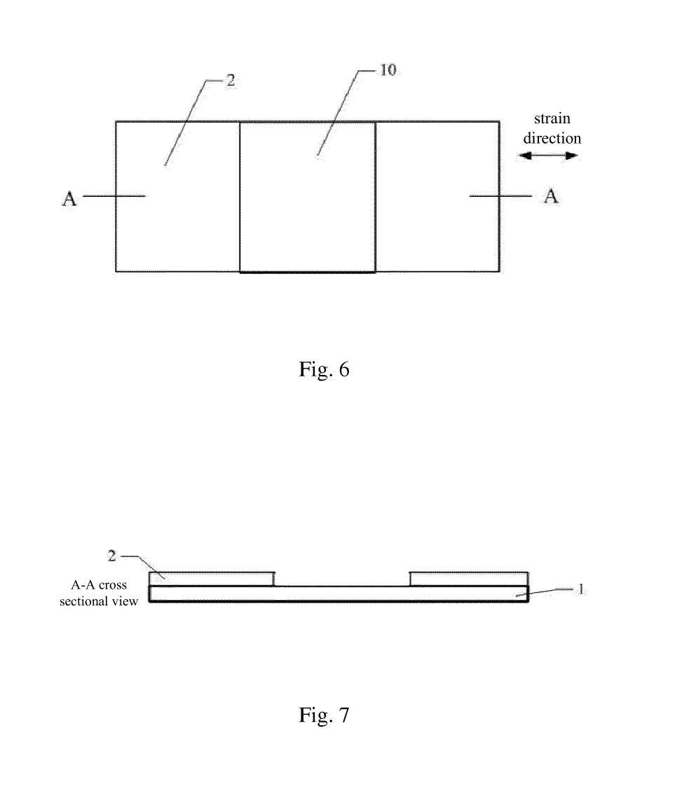

Furthermore, referring to FIG. 6 and FIG. 7, they indicate a specific composition of a flexible textile sensor according to one embodiment of the invention. The flexible textile sensor includes a flexible textile layer 1 which has a sensing stress, a fixed layer 2 which is bond on the flexible textile layer. There is no fixed layer bond on the stress-sensing domain 10 of the flexible textile layer 1, and the fixed layer 2 can be bond on the fixed domain besides the stress-sensing domain 10 of the flexible textile. The strain of the fixed layer can be less than the strain of flexible textile, and the fixed layer above can be an inelastic mechanical textile.

Since the strain of the fixed layer is less than the strain of the flexible textile, when the flexible textile is deformed due to the strain stress, it can be ensured that the deformation can only occur in the stress-sensing domain on which there is no binding fixed layer. In this way, the size of the stress-sensing domain can be controlled accurately by controlling the domain of the flexible textile without a covered bonding fixed layer. And thus, the accurate measure of the stress can be realized.

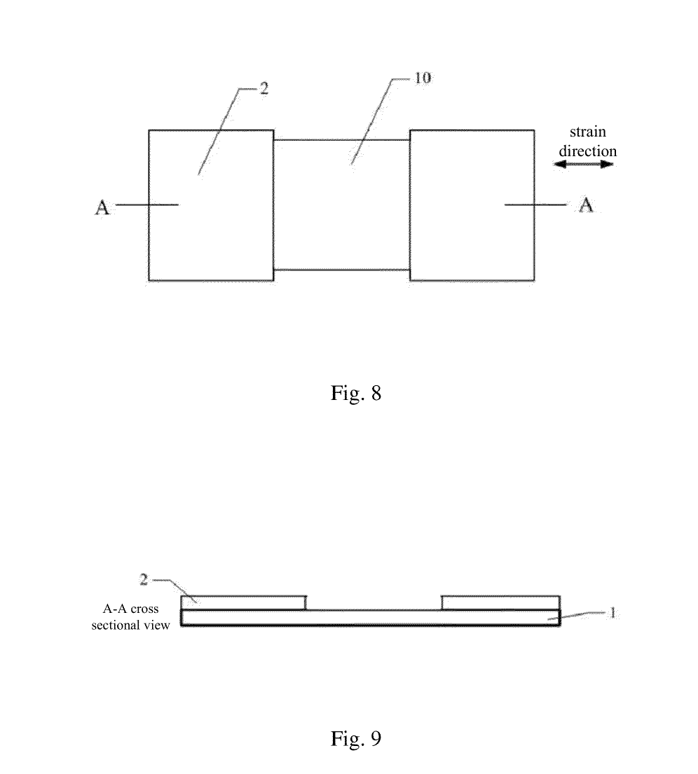

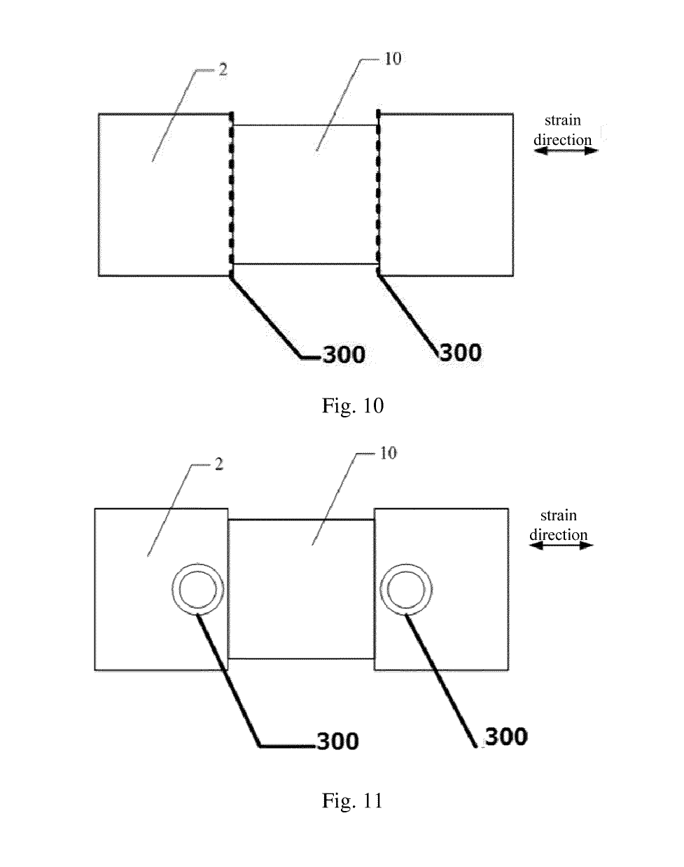

The forms of the stress-sensing domain shown in FIG. 6 and FIG. 7 can only be used for an illustration. Other forms can also be applied, such as the ones shown in FIG. 8 and FIG. 9, which indicate a composition of another flexible textile sensor according to one embodiment of the invention.

Referring to FIG. 10, it illustrates one of electrical wire connection manners on the flexible textile according to one embodiment of the invention. In one embodiment of the invention, sewing the connecting electrical wire 300 can be used, and other connection manners of electrical wires can also be used, for example, the connecting electrical wire or the conductive button can be bond, mechanically clamped, sewn on the stress-sensing domain in the strain direction, in another way, the connecting electrical wire or the conductive button can be bond, mechanically clamped, sewn on the fixed domain at the double sides of the stress-sensing domain in the strain direction. Referring to FIG. 11, it is an illustration of mechanically clamping the conductive button on the fixed domain at the double sides of the stress-sensing domain in the strain direction. In one embodiment of the invention, the conductive button can be electrically connected with the electrical wire 300. The conductive button can be designed as the form of concavity or convexity. The concave and the convex buttons can be locked when the measurement is required, and can be separated when no measurement is required. FIG. 11 shows the scenario of the buttons being locked.

During the practical implementation, the following situation has been discovered: the strain generally comes from the two corresponding sides, which leads to the conductive end of the sensor usually placed at the double sides of the stress-sensing part in the strain direction. And thus it is unbeneficial to layout the follow-up circuit of the sensor, which means the electrical wire in one of the two ends will always need to be placed in the strain direction.

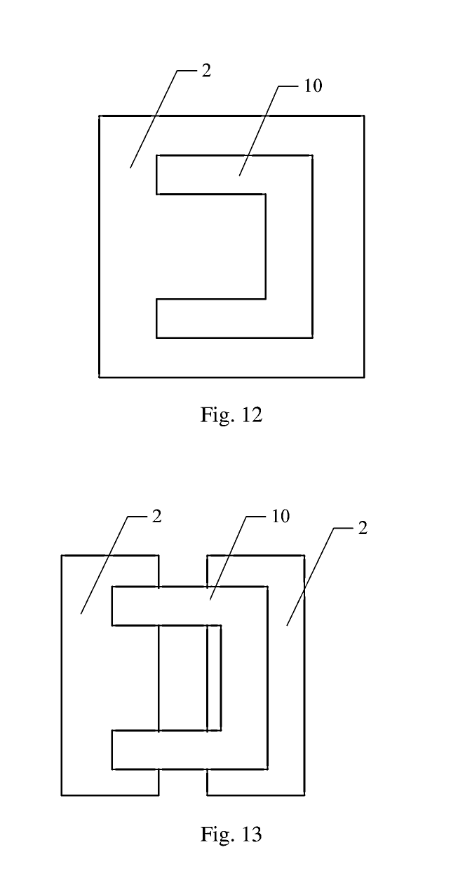

Referring to FIG. 12 and FIG. 13, they illustrate another composition of a flexible textile sensor according to one embodiment of the invention. The flexible textile sensor includes a flexible textile layer having a sensing stress, and a fixed layer which is bond on the flexible textile layer. The textile of the flexible textile layer can form a C shape, the upper arm and the lower arm of the C shape can be the stress-sensing domain, and the fixed layer can be fixed on the starting point side, the ending point side, and the left-bending side of the C shape. The strain of the fixed layer is less than the strain of the flexible textile. The connecting electrical wire or the conductive button can be bond, mechanically clamped, sewn on the both sides of the starting point and the ending point of the C shape. The fixed layer can be an inelastic mechanical textile.

In this way, when the conductive end and the follow-up circuit are designed, both of them can be designed at one side of the strain direction, and thus the interruption from the conductive circuit to the stress measurement can be reduced. Considering of that, the textile shape of the flexible textile layer above can form other shapes like the C shape, such as a U shape which rotates 90 degrees, V shape and so on. It can be applied to all the shapes, which can not only realize the strain measurement of the stress direction, but also can make both conductive ends placed at the same side of the stress direction.

When the flexible textile sensor according to one embodiment of the invention is produced, the semi-finished product can include the following structures: the flexible textile layer having a sensing stress, and a fixed layer bond on the flexible textile layer. The fixed layer can have a hollow pattern, and the hollow part of the hollow pattern can correspond to the stress-sensing domain of the flexible textile. The strain of the fixed layer can be less than the strains of the flexible textile.

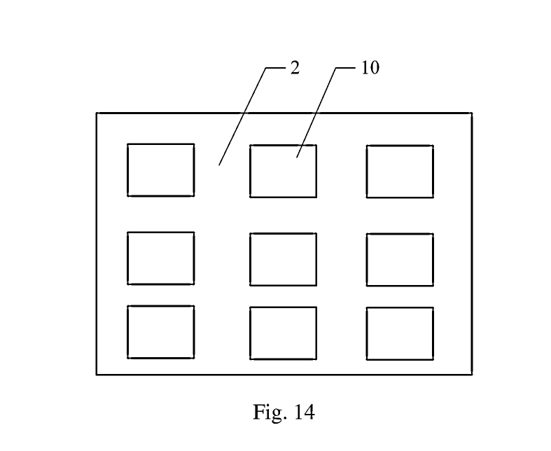

It means that in the process of producing the sensor, a relative large size of the flexible textile can be made at one time, and it can be bond with the hollow fixed layer to form a double-layer structure. With an appropriate design of the hollow pattern of the fixed layer, multiple stress-sensing parts of the flexible textile sensor can be obtained by tailoring the double-layer structure. Referring to FIG. 14, it is an illustration of the hollow pattern, particularly, the shape, size and the interval of the hollow pattern can be designed and modified according to the realistic requirement.

Meanwhile, in the specific implementation, the hollow pattern shown in FIG. 14 can be designed as a C shape or the like shape.

In one embodiment of the invention, little elastic or no elastic fixed layer can be bond on the flexible textile layer which has the elastic sensing stress of the flexible textile sensor, and no fixed layer will be bond on the stress-sensing domain of the flexible textile. It can be ensured that the stress-sensing domain of the flexible textile can sense the stress, and only little or no deformation may occur at other domains of the flexible textile. Therefore, it can be beneficial for the flexible textile to connect with other parts of the whole sensing system. In one embodiment of the invention, the pressure monitoring shoe can be applied for: (1) somatosensory game, as a prop shoe; (2) different kinds of sports shoes, such as a golf sports shoe, a basketball shoe, a football shoe, a shadow boxing shoe, and so on; (3) a weight monitoring shoe, for real-time monitoring the weight of the monitoring object during the weight reduction process; (4) a recuperation monitoring shoe, for monitoring a recuperation for a stroke patient, or the recuperation for an ankle, a leg or the like, and other situations.

The above descriptions are some exemplary embodiments of the invention, and should not be regarded as limitation to the scope of related claims. A person having ordinary skills in a relevant technical field will be able to make improvements and modifications within the spirit of the principle of the invention. The improvements and modifications should also be incorporated in the scope of the claims attached below.

1. A pressure monitoring shoe, comprising a main shoe body, a data processing device, and at least two pressure sensors;
wherein, the pressure sensors are arranged at one or a plurality of locations where the main shoe body is in contact with a foot; and
the data processing device is arranged in one or more of a sole space, an outside space of the shoe body or a rear space of the heel of the pressure monitoring shoe, and a signal connection line is arranged between the data processing device and the pressure sensor.

2. The pressure monitoring shoe of claim 1, wherein the pressure sensor comprises a flexible textile sensor and an encapsulation device which for encapsulating the flexible textile sensor, or the pressure sensor is denoted as a pressure sensor of resistance type, capacitive type, inductance type.

3. The pressure monitoring shoe of claim 1, wherein the flexible textile sensor comprises a flexible textile layer having a sensing stress, a fixed layer bond on the flexible textile; no fixed layer is bond on a stress-sensing domain of the flexible textile, and the fixed layer is bond on a fixed domain besides the stress-sensing domain; a strain of the fixed layer is less than the strain of the flexible textile.

4. The pressure monitoring shoe of claim 3, wherein the fixed layer is an inelastic mechanical textile.

5. The pressure monitoring shoe of claim 3, wherein the flexible textile layer forms a C shape, and an upper arm and a lower arm of the C shape are the stress-sensing domains; the fixed layer is fixed on a starting point side, an ending point side, and a left-bending side of the C shape; the strain of the fixed layer is less than the strain of the flexible textile.

6. The pressure monitoring shoe of claim 1, wherein the data processing device comprises a transmitting module, which is configured to receive a signal generated by the pressure sensor and transmit the signal to an external data receiving platform by a wired or a wireless mode.

7. The pressure monitoring shoe of claim 1, wherein the data processing device comprises a data storage module, which is configured to receive and storage a signal generated by the pressure sensor, convert the signal generated by the pressure sensor into a pressure data, and storage the pressure data.

8. The pressure monitoring shoe of claim 2, wherein the encapsulation device comprises a flexible shell body, the pressure sensor is encapsulated in the flexible shell body and fixed on a bottom substrate of the shell body, the flexible shell body is stretched with the flexible textile sensor being stretched and compressed with the flexible textile sensor being compressed; or
the encapsulation device comprises a flexible coating, the flexible coating is coated on all sides of the pressure sensor, and the flexible coating is stretched with the flexible textile sensor being stretched and compressed with the flexible textile sensor being compressed.

9. The pressure monitoring shoe of claim 3, wherein a connecting electrical wire or a conductive button is bond, mechanically clamped, sewn on double sides of the stress-sensing domain in the strain direction.

10. The pressure monitoring shoe of claim 3, wherein the connecting electrical wire or the conductive button is bond, mechanically clamped, sewn on the fixed domain at the double sides of the stress-sensing domain in the strain direction, and the connecting electrical wire or the conductive button is conductively connected with the flexible textile.

11. The pressure monitoring shoe of claim 1, further comprising a charging module in the main shoe body, and the charging module is configured to charge for the pressure sensor and the data processing device.

12. The pressure monitoring shoe of claim 11, wherein the charging module is any one of a disposable un-chargeable battery, a chargeable battery, a mechanically charged device.
